(19) United States
(12) Patent Application Publication
(10) Pub. No.: US 2003/0105167 A1
(43) Pub. Date: Jun. 5, 2003

(54) Treatment of opthalmic diseases

(76) Inventors: James Alan Dykens, Encinitas, CA (US); Katherine Gordon, Winchester, MA (US)

Correspondence Address: BROMBERG & SUNSTEIN LLP, 125 SUMMER STREET, BOSTON, MA, 02110-1618, US

(21) Appl. No.: 10/313,172

(22) Filed: Dec. 5, 2002

Related U.S. Application Data
(60) Provisional application No. 60/336,599, filed on Dec. 5, 2001.

(51) Int. Cl. A61K 31/05

(52) U.S. Cl. 514/732

(57) ABSTRACT

The present invention relates to a method of using protective compounds for the prevention or treatment of ophthalmic diseases, disorders or injuries in a subject. The method comprises the step of administering a predetermined polycyclic phenolic compound to a subject in need thereof. The polycyclic phenolic compound is selected from those having at least one terminal phenolic group and at least one other cyclic group.

### TECHNICAL FIELD AND BACKGROUND

Mitochondria are the cellular components that generate over 90% of the cell's energy, and at the same time also generate over 90% of the deleterious free radicals to which cells are exposed. As such, even modest impairment of normal mitochondrial function imposes an energetic and oxidative stress on the cell that, if left unchecked, presages cell death. Indeed, acute loss of mitochondrial function causes necrotic cell death, whereas moderate loss-of mitochondrial function initiates cell death via apoptosis, or “cell suicide”. This is the case for all aerobically poised cell types, including many of the cells found in the eye. For example, loss of transparency in the lens is associated with radical-mediated cross linking of the lens proteins, and the apoptotic death of retinal ganglion cells in glaucoma is induced by mitochondrial failure.

Primary open-angle glaucoma (POAG) is a leading cause of blindness in the United States and its prevalence is expected to increase as the population ages. The disease is typically treated by reducing the major risk factor for the disease, elevated intraocular pressure (IOP), with appropriate pharmaceutical regimes and sometimes surgical techniques such as trabeculotomy. Current therapeutics, including beta-blockers, alpha-2 agonists, prostanoids and carbonic anhydrase inhibitors, lower IOP by decreasing the production of aqueous humor, for example. Decreasing the intraocular pressure protects the optic nerve indirectly by preventing further pressure-induced mechanical or ischemic damage. However, many patients still manifest glaucomatous visual loss due to progressive neuronal damage despite the normalization of intraocular pressure.

The visual loss that results from elevated IOP is caused by the death of retinal ganglion cells (RGCs) and loss of nerve fiber layer (NFL) in the retina, secondary to optic nerve damage. This death may be precipitated by decreased nutrition (or decreased trophic factors) caused by ischemia, pressure-induced, or injury-induced reduction of retrograde axoplasmic transport. Recent consensus seems to be that progression of retinal and optic neuronal loss occurs even when the primary source of the damage, such as elevated IOP, is removed (Schwartz et al., 1996). The evidence indicates that glaucomatous loss of RGCs results from both necrotic and apoptotic processes (Nickells, 1999; Tatton et al., 2001a,b; Hartwick, 2001; Weinreb and Levin, 2002). As such, modern views of glaucoma characterize it as a neurodegenerative disease, essentially an optic neuropathy, in which degeneration of the retinal ganglion cells and their axons results in a characteristic excavation in the optic disc (Weinreb and Levin, 2002). There are no neuroprotective therapeutics currently available for RGC death in glaucoma, or for cytoprotection in other ocular tissues, (Schwartz et al., 1996; Nickells, 1999; Weinreb and Levin, 1999; Levin, 2001), underscoring the need for safe and effective neuroprotective treatments that can be used on a chronic basis in both genders.

As is the case for other cell types in the eye and elsewhere, RGC death may be precipitated by a variety of toxic stressors, including decreased neurotrophic support (Yuan and Neufeld, 2000; Schmeer et al., 2002), excessive expression of nitric oxide synthase (Neufeld, 1999), overexposure to excitatory dicarboxylate amino acids such as glutamate (Hartwick, 2001; Hare et al., 2001), or other injuries such as increased hydrostatic pressure or reduction of retrograde axoplasmic transport (Dreyer, 1998; Brooks et al., 1999; Vorwerk et al., 2000). Regardless of the initial insult(s), the cellular and mitochondrial mechanisms that precipitate RGC death include increased free radical production, and loss of both ionic and energetic homeostasis. This etiology is partly analogous to the death of neurons known to occur in chronic neurodegenerative diseases such as Alzheimer's and Parkinson's disease, and partly analogous to the excitotoxic and apoptotic cell death that occurs in ischemic stroke (reviewed by Dykens, 1997; Manfredi and Beal, 2000; Hartwick, 2001; Tatton et al., 2001 a,b).

Excitotoxicity results from excessive stimulation of dicarboxylate receptors, including the NMDA receptor and other voltage-gated and metabotropic receptors, by excitatory neurotransmitters, such as glutamate and quinolinate. The resulting acute elevation in cytosolic Ca⁺² destabilizes mitochondria which not only accelerates free radical production, but also undermines cellular energy status, both of which conspire to kill the cell via necrosis or apoptosis. Mitochondria are thus key modulators of neuronal viability under a wide variety of injurious circumstances implicated in glaucomatous RGC death, including oxidative insult, energetic impairment, ionic and osmotic failure, plus other cytotoxic processes (Hartwick, 2001; Shori et al., 2001). As such, mitochondria provide a critical point for potential therapeutic intervention; to the extent that energetic and oxidative pathologies conspire to undermine RGC integrity, mitochondrially directed intervention should prove beneficial. Tatton has proposed that deprenyl and its primary metabolite desmethyldeprenyl (DES) which in part maintain mitochondrial membrane integrity may prove useful in the treatment of glaucoma (Tatton, 1999; Tatton et al., 2001a,b patents 6,455,590; 5,981,598; 5,783,606).

Retinal or optic nerve ischemia or hypoxia results when blood supply is significantly reduced to these tissues. Ischemia is a complex pathological episode involving numerous biochemical events, not the least of which is diminished mitochondrial function resulting from insufficient vascular delivery of oxygen. In recent years, excitatory amino acids in ischemia-related neuronal and retinal damage have been implicated. (See, e.g., Choi, Excitatory cell death, Journal of Neurobiology, volume 23, pages 1261-1276 (1992)). In addition, the production of free radicals by mitochondria exposed to excitotoxic conditions has been directly demonstrated (Dykens, J. A., (1994). Isolated cerebellar and cerebral mitochondria produce free radicals when exposed to elevated Ca⁺² and Na⁺: Implications for Neurodegeneration, J. Neurochemistry, Vol. 63, pp. 584-591). Compounds which perturb mitochondrial function, such as ethambutol, have been shown to be toxic to retinal ganglion cells by an excitotoxic pathway (Heng et al., (1999) Invest Ophthalmol Vis Sci., Vol. 40, pp. 190-6).

Diabetic retinopathy is an ophthalmic disease leading to loss of vision and even blindness. It has been reported that glutamate excitotoxicity has played a role in such vision loss. (See, e.g., Ambati, et al., (1997) Elevated GABA, Glutamate, and VEGF in the Vitreous of Humans with Proliferative Diabetic Retinopathy, Invest. Ophthalmol. Vis. Sci., Vol. 38, pp. S771). This reference suggested that high levels of glutamate are potentially toxic to retinal ganglion cells.

17β-Estradiol, the naturally-occurring hormone, has recently been shown to preserve mitochondrial function in the presence of an oxidative phosphorylation uncoupler, 3-nitroproprionic acid (Wang et al., (2001) J. Neurochem, Vol. 77, pp. 804-811). This mechanism may be an important component of estrogen's neuroprotective effects which have been widely described against a variety of toxicities, including growth factor deprivation, glutamate toxicity, and oxidative stress. Similarly, in rodents, 17β-estradiol has been shown to attenuate neuronal loss after the induction of cerebral (Simpkins et al., (1997) J. Neurosurg, Vol. 87, pp. 724-730; Yang et al., (2000) Stroke, Vol. 31, pp. 70-75) and retinal (Nonaka et al., (2000) Invest Ophthalmol Vis. Sci., Vol. 41, pp. 2689-2696) ischemia, both of which may entail glutamate excitotoxicity and mitochondrial dysfunction leading to apoptosis in the penumbra of the stroke.

17β-estradiol has also been shown to function as a neuroprotectant against retinal ischemia in vivo (Nonaka, et al., 2000). In this study, retinal ischemia was induced in rats by ligation of the optic nerve, and 17β-estradiol was administered prior to the insult. Several days following reperfusion, cell density in the retinal ganglion layer was significantly higher in animals that had been treated with estradiol. Although these experiments establish proof-of-principal with estradiol, this compound is not an ideal therapeutic candidate because of the ancillary hormonal activities.

Several lines of evidence suggest that the neuroprotective effects of estrogens do not require classical estrogen-receptor (ER) dependent gene transcription. Several so-called “non-hormonal estrogens”, 17α-estradiol (Green et al., 1997 J. Neurosci., Vol. 17, pp. 511-515; Green et al 1997 J. Steroid Biochm. Mol. Biol., Vol. 63, pp. 229-235) and ent-estradiol (Green et al., (2001) Endocrinology, Vol. 142, pp. 400-406) for instance, are highly potent neuroprotectants even though they do not bind ER or stimulate estrogen-responsive tissues. Further, functional ERs have not been found in either HT-22 cells, or SK-N-SH cells, although numerous studies demonstrate estrogen-mediated protection of these neuronal cell lines. See Behl et al., (1997) Mol Pharmacology, Vol. 51, pp. 535-541; Zhang et al., (1998) Brain Res, Vol. 784, pp. 321-324; Green et al., (1998) Neuroscience, Vol. 84, pp. 7-10. Similarly, 17α-E2-mediated protection can occur in the presence of ER antagonists. These studies implicate cellular mechanisms other than classical ER activity in the neuroprotective effects of estrogens, while not excluding a role for ERs in neuroprotection. Structure-activity studies have defined the minimal requirement for neuroprotection to be the presence of a phenolic A ring and have led to the development of novel polycyclic phenolic compounds (PPCs).

Although the exact mechanisms have not been worked out, it is clear that various forms of estrogen are capable of lowering IOP in humans. For instance, continuous oral treatment of normal women with mestranol causes a gradual decrease in IOP (Treister et al., 1970). Oral administration of mestranol with norethynodrel to patients with primary open angle glaucoma reduces IOP (Meyer et al., 1966). More recent studies have shown that menopause is associated with a significant increase in IOP (Qurshi, 1996), implying that the premenopausal hormones are involved in regulating trabecular meshwork homeostasis. Also, in a post-menopausal glaucoma patient, hormone replacement was been shown to reduce IOP (Sator et al., 1998).

As previously mentioned, normalization of IOP is not sufficient to protect against the onset or progression of disease in the case of glaucoma. Many patients being treated with IOP-lowering drugs believe erroneously that they are protected from the blindness due to glaucoma. An optimal therapeutic would minimize or mitigate risk factors while protecting cells or tissues from the consequences of damage. Unfortunately, current therapies to lower IOP can also induce apoptosis of the trabecular meshwork cells while lowering the risk factor for glaucoma.

Treatments for retinal diseases, macular degeneration and glaucoma include compounds with anti-oxidant and NMDA antagonist activity (U.S. Pat. No. 6,200,990), polyamine antagonists (U.S. Pat. No. 5,604,244), calpain inhibitors (US 6,303,579), estrogen metabolites (U.S. Pat. No. 5,521,168) and carvedilol (U.S. Pat. No. 6,291,506).

### SUMMARY OF THE INVENTION

The current invention centers around the unexpected observation that non-hormonal polycylic phenolic compounds (“PPCs”) stabilize mitochondria in retinal tissue under conditions of cellular stress such as excessive calcium load. Although the PPCs are known to be cytoprotective, action at the mitochondria renders these compounds even more potently protective and points to specific therapeutic modalities such as protection of ophthalmic tissue in ophthalmic diseases in the presence of compromising mitochondrial toxins.

We have tested this by assessing the effects of PPCs on mitochondrial function and viability of primary RGCs and, a transformed RGC line exposed to pathologically relevant stressors.

PPCs ideally do not interact with either of the classical estrogen receptors, and therefore lack hormonal action, an important consideration for chronic treatment of both genders. Our studies show that non-hormonal PPCs are able to stabilize mitochondrial function in retinal ganglion cells under pathologically relevant conditions of calcium loading.

In one exemplary embodiment of the invention there is provided a method of using protective compounds for the prevention or treatment of ophthalmic diseases, disorders or injuries in a subject.

Another exemplary embodiment of the invention provides a method of using protective compounds for the prevention or treatment of ophthalmic diseases, disorders or injuries in a subject wherein the protective compound protects against cell death.

Still another exemplary embodiment of-the invention provides a method of using protective compounds for the prevention or treatment of ophthalmic diseases, disorders or injuries in a subject wherein the cell death occurs by apoptosis.

A further exemplary embodiment of the invention provides a method of using protective compounds for the protection of retinal ganglion cells in a subject at risk of retinal ganglion cell damage.

Yet another exemplary embodiment of the invention describes administering protective compounds to a subject in conjunction with an administration of a medical treatment suspected of destabilizing mitochondrial function in retinal ganglion cells.

Still a further exemplary embodiment of the invention discloses a method of preventing or treating ophthalmic diseases, disorders or injuries in a subject, comprising selecting a compound shown to stabilize or enhance mitochondrial survival or activity and administering said compound to a subject in need thereof.

Another exemplary embodiment of the present invention describes a method for preventing or treating ophthalmic diseases, disorders or injuries in a subject according to which a protective compound is administered to said subject, wherein the protective compound acts by a dual mechanism, the dual mechanism comprising normalizing intraocular pressure as well as directly protecting against cell death.

In yet a further exemplary embodiment of the invention, a method is described wherein a protective compound is administered prophylactically to at-risk patients not yet showing signs of an ophthalmic disease, disorder or injury, thereby preventing or delaying onset of the disease.

In still a further aspect of the invention, a protective compound is administered to a subject, wherein the subject is additionally treated with one or more medications known to perturb mitochondria and/or medications known to cause optic neuropathy.

A further exemplary embodiment of the invention provides a pharmaceutical composition for preventin g or treating an ophthalmic disease, disorder or injury in a subject, comprising an effective dose of a protective agent in a suitable formulation.

### BRIEF DESCRIPTION OF THE DRAWINGS

Exemplary embodiments of the present invention are described hereinafter and may be better understood with reference to the appended Figures in which:

FIG. 1 demonstrates that 17α- and 17β-estradiol stabilize mitochondrial membrane potential (Δψₘ) in SHSY-5Y neuroblastoma cells against Ca⁺²-induced collapse; and

FIG. 2 demonstrates that PPCs moderate Ca⁺² induced ΔΨₘ collapse in neuroblastoma and retinal ganglion cells.

### DETAILED DESCRIPTION OF SPECIFIC EMBODIMENTS

Definitions. As used in this description and the accompanying claims, the following terms shall have the meanings indicated, unless the context otherwise requires: “Ophthalmic diseases, disorders or injuries” includes: diabetic retinopathy, glaucoma, macular degeneration, retinitis pigmentosa, retinal tears or holes, retinal-detachment, retinal ischemia, acute retinopathies associated with trauma, inflammatory mediated degeneration, post-surgical complications, damage associated with laser therapy including photodynamic therapy (PDT), surgical light induced iatrogenic retinopathy, drug-induced retinopathies, autosomal dominant optic atrophy, toxic/nutritional amblyopias; Leber's Hereditary Optic Neuropathy (LHOP), other mitochondrial diseases with ophthalmic manifestations or complications, Angiogenesis; Atypical RP; Bardet-Biedl Syndrome; Best Disease; Blue-Cone Monochromacy; Cataracts; Central Areolar Choroidal Dystrophy; Choroideremia; Cone Dystrophy; Rod Dystrophy; Cone-Rod Dystrophy; Rod-Cone Dystrophy; Congenital Stationary Night Blindness; Cytomegalovirus Retinitis; Diabetic macular edema; Dominant Drusen; Giant Cell Arteritis (GCA); Goldmann-Favre Dystrophy; Graves' Ophthalmopathy; Gyrate Atrophy; Hydroxychloroquine; Iritis; Juvenile Retinoschisis; Kearns-Sayre Syndrome; Lawrence-Moon Bardet-Biedl Syndrome; Leber Congenital Amaurosis; Lupus-induced Cotton Wool Spots; Macular degeneration, dry form; Macular degeneration, wet form; Macular Drusen; Macular Dystrophy; Malattia Leventinese; ocular histoplasmosis syndrome; Oguchi Disease; Oxidative damage; Proliferative Vitreoretinopathy; Refsum Disease; Retinitis Punctata Albescens; retinopathy of prematurity; Rod Monochromatism; RP and Usher syndrome; Scleritis; Sector RP; Sjogren-Larsson Syndrome; Sorsby Fundus Dystrophy; Stargardt Disease and other retinal diseases.

Polycyclic phenolic compounds (“PPCs”) are compounds which have at least one terminal phenolic ring and at least one other terminal carbon cyclic group and their salts, isomers, enantiomers, prodrugs and precursors. Generally, PPCs having the required activity will have 2, 3, 4, and even 5 ring structures, although they will generally have a molecular weight less than 2000 Daltons, preferably less than 1500 Daltons and more preferably less than 1000 Daltons. Examples of PPCs believed to be of utility in exemplary embodiments of the present invention are found in PCT Publications WO02/36605 and WO00/63228 as well as in several of the US patents incorporated herein by reference further below.

“Protective compounds” includes polycyclic phenolic compounds having a terminal phenolic group in a structure containing at least a second ring. Examples of such compounds are described in U.S. Pat. Nos. 5,554,601, 5,859,001, 5,972,923 and 6,197,833B 1, all incorporated herein by reference. Protective compounds generally, although not always, have a molecular weight of less than 1000 Daltons. The effective dose of a protective compound generally provides a tissue or blood concentration of the compound that is equal to or less than 500 nM. Although some of the referenced patents disclose examples of estrogen-based compounds as well as non-estrogen compounds, as mentioned hereinabove, it has been observed that the protective effect of these compounds is independent of their ability to bind the classical ERa and ERP estrogen receptors. With respect to estrogen-based compounds, it is, in fact, preferable to use non-hormonal estrogen analogs, since the hormonal-aspects of estrogens are most often not needed, and can even be undesirable. Hormonal estrogens such as 17β-estradiol and its hormonal analogs might be used in exemplary embodiments of the invention, but preferably only in circumstances where their retino-protective properties are so high as to either {a} permit them to be used-in sufficiently small quanitities so that their hormonal effects are insubstantial or {b } they are primarily administered to female patients or {c} the hormonal effects are otherwise attenuated, such as by the method of administration, for example in eye drops.

By “non-hormonal” estrogen it is meant an estrogen analog which, regardless of its ability to bind the classical ERa and ERP estrogen receptors, still do not elicit the receptor mediated hormonal effect normally associated with hormonal estrogen.

The current invention centers around the unexpected observation that non-hormonal polycylic phenolic compounds (“PPCs”) stabilize mitochondria in retinal tissue under conditions of cellular stress such as excessive calcium load. Although the PPCs are known to be cytoprotective, action at the mitochondria renders these compounds surprisingly potent in their protective abilities and points to specific therapeutic modalities such as protection of ophthalmic tissue in ophthalmic diseases in the presence of compromising mitochondrial toxins. Although PPCs can include compounds having hormonal properties analogous to estrogen, many PPCs, and even many estrogen analogs, ideally do not interact with either of the classical estrogen receptors, and therefore lack hormonal action, an important consideration for chronic treatment of both genders. Our studies show that non-hormonal PPCs are able to stabilize mitochondrial function in retinal ganglion cells under pathologically relevant conditions of calcium loading.

Exemplary embodiments of the invention include the use of a protective compound having a terminal phenolic ring and at least a second carbon ring.

In addition to these required structures, the compound may have a number of R groups attached to any available site on the phenolic ring or elsewhere providing that the phenolic structure of the terminal ring is maintained. These R-groups may be selected from inorganic or organic atoms or molecules. Non-limiting examples of a number of different types of R groups include any inorganic R group including any of a halogen, an amide, a sulfate, a nitrate, fluoro, chloro, or bromo groups. Additionally, R groups selected from sodium, potassium and/or ammonium salts may be attached to the alpha or beta positions to replace hydrogen on any available carbon in the structure. The R-group may be organic or may include a mixture of organic molecules and ions. Organic R groups may include alkanes, alkenes or alkynes containing up to six carbons in a linear or branched array. For example, additional R group substituents may include methyl, ethyl, propyl, butyl, pentyl, hexyl, heptyl, dimethyl, isobutyl, isopentyl, tert-butyl, sec-butyl, isobutyl, methylpentyl, neopentyl, isohexyl, hexenyl, hexadiene, 1,3-hexadiene-5-yne, vinyl, allyl, isopropenyl, ethynyl, ethylidine, vinylidine, isopropylidene; methylene, sulfate, mercapto, methylthio, ethylthio, propylthio, methylsulfinyl, methylsulfonyl, thiohexanyl, thiobenyl, thiopenol, thiocyanato, sulfoethylamide, thionitrosyl, thiophosphoryl, p-toluenesulfonate, amino, imino, cyano, carbamoyl, acetamido, hydroxyamino, nitroso, nitro, cyanato, selecyanato, arccosine, pyridinium, hydrazide, semicarbazone, carboxymethylamide, oxime, hydrazone, sulfurtrimethylammonium, semicarbazone, o-carboxymethyloxime, aldehyde hemiacetate, methylether, ethylether, propylether, butylether, benzylether, methylcarbonate, carboxylate, acetate, chloroacetate, trimethylacetate, cyclopentylpropionate, propionate, phenylpropionate, carboxylic acid methylether, formate, benzoate, butyrate, caprylate, cinnamate, decylate, heptylate, enanthate, glucosiduronate, succinate, hemisuccinate, palmitate, nonanoate, stearate, tosylate, valerate, valproate, decanoate, hexahydrobenzoate, laurate, myristate, phthalate, hydroxyl, ethyleneketal, diethyleneketal, formate, chloroformate, formyl, dichloroacetate, keto, difluoroacetate, ethoxycarbonyl, trichlorofornate, hydroxymethylene, epoxy, peroxy, dimethyl ketal, acetonide, cyclohexyl, benzyl, phenyl, diphenyl, benzylidene, and cyclopropyl groups. R groups may be attached to any of the constituent rings to form a pyridine, pyriazine, pyrimidine, or v-triazine. Additional R group substituents may include any of the six member or five member rings itemized below.

Protective compounds may also be selected from those having in addition to the phenol A ring, a heterocyclic carbon ring which may be an aromatic or non-aromatic phenolic ring with any of the substitutions described above and further may be selected from, for example, one or more of the following structures phenanthrene, naphthalene, napthols, diphenyl, benzene, cyclohexane, 1,2-pyran, 1,4-Pyran, 1,2-pyrone, 1,4-pyrone, 1,2-dioxin, 1,3-dioxin (dihydro form), pyridine, pyridazine, pyrimidine, pyrazine, piperazine, s-triazine, as- triazine, v-triazine, 1,2,4-oxazine, 1,3,2-oxazine, 1,3,6-oxazine (pentoxazole), 1,2,6 oxazine, 1,4-oxazine, o-isoxazine, p-isoxazine, 1,2,5-oxathiazine, 1,2,6-oxathiazine, 1,4,2-oxadiazine, 1,3,5,2-oxadiazine, morpholine (tetrahydro-p-isoxazine), any of the six ringed structures listed above being a terminal group in the compound. Additionally, any of the above carbon ring structure may be linked directly or via a linkage group to any further heterocyclic aromatic or non aromatic carbon ring including: furan; thiophene (thiofuran); pyrrole (azole); isopyrrole (isoazole); 3-isopyrrole (isoazole); pyrazole (1,2-daizole); 2-isoimidazole (1,3-isodiazole); 1,2,3-triazle; 1,2,4 triazole; 1,2-diothiole; 1,2,3-oxathiole, isoxazole (furo(a) monozole); oxazole (furo(b) monazole); thiazole; isothiazole; 1,2,3-oxadiazole; 1,2,4-oxadiazole, 1,2,5-oxadiazole, 1,3,5 oxadiazole, 1,2,3,4-oxatiazole; 1,2,3,5-oxatriazole; 1,2,3-dioxazole; 1,2,4-dioxazole; 1,3,2-dioxazole; 1,3,4-dioxazole; 1,2,5-oxathiazole; 1,3-oxathiole, cyclopentane. These compounds in turn may have associated R groups selected from the groups described above that are substituted on the carbon ring at any of the available sites.

Exemplary embodiments of protective compounds may include any compound, including those listed above, that may form a cyclopentanophen(a)anthrene ring compound and which, for example, may be selected from the group consisting of 1,3,5 (10), 6,8-estrapentaene, 1,3,5 (10), 6,8, 1 1-estrapentaene, 1,3,5 (10) 6,8,15-estrapentacne, 1,3,5 (10), 6,-estratetraene, 1,3,5 (10), 7-estratetraene, 1,3,5 (10)8-estratetraene, 1,3,5 (10)16-estratetraene, 1,3,5 (10)15-estratetraene, 1,3,5 (10)- estratriene, 1,3,5 (10) 15-estratriene.

Further exemplary embodiments of protective compounds may include any compound including precursors or derivatives selected from raloxifen, tamoxifen, androgenic compounds, and their salts where an intact phenol ring is present with a hydroxyl group present on carbons 1,2,3 and 4 of the terminal phenol ring.

Additional exemplary embodiments of protective compounds useful in the invention include any compound in the form of a prodrug, that may be metabolized to form an active polycyclic phenolic compound having neuroprotective activity, as well as its salts, isomers, eniantiomers and pharmaceutical formulations of the above.

An “estrogen compound” is defined here and in the claims, per the description in U.S. Pat. No. 5,554,601, incorporated herein by reference, as any of the structures described in the 11th edition of “Steroids” from Steraloids Inc., Wilton N. H., here incorporated by reference. Other estrogen compounds included in this definition are estrogen derivatives, estrogen metabolites and estrogen precursors as well as those molecules capable of binding cell-associated estrogen receptor as well as other molecules where the result of binding specifically triggers a characterized estrogen effect. Sub-categories included in this definition are non-steroidal estrogens described in the aforementioned references, non-hormonal estrogens, hormonal estrogens. As mentioned hereinabove, by non-hormonal estrogens is meant estrogen forms which exert a protective effect without hormonal activation of classical E-α and/or classical E-β estrogen receptors. Examples of estrogen structures having utility either alone or in combination with other agents are provided in FIG. 9 of U.S. Pat. No. 5,554,601, incorporated herein by reference.

Current therapies to lower IOP, while lowering the risk factor for glaucoma, can also induce apoptosis of the trabecular meshwork cells. Thus, the application of the cytoprotective drugs in accordance with exemplary embodiments of the invention are needed that not only can reduce risk, but also protect the target cells from damage.

Accordingly, protective compounds may be used in the prevention or treatment of ophthalmic diseases, ophthalmic disorders or ophthalmic injury.

The protective compounds are selected according to their mode of action. For example; (a) Protective compounds are selected which act by protecting retinal ganglial cells and other tissues in the eye from cell death. The cell death mechanism may include an apoptotic process or mitochondrial dysfunction; (b) Protective compounds are selected according to their ability to effectively augment the activity and survival of mitochondria, deficiencies of which lead to depletion of metabolic active transport mechanisms cellular ATP levels, and apoptotic cell death; (c) Protective compounds are selected according to their ability to effectively give rise to a dual mechanism, normalizing intraocular pressure as well as directly protecting against cell death, thereby providing optimal protection against the onset and progression of the ophthalmologic disease.

The protective compounds may be used prophylactically or therapeutically in at-risk patients identified symptomatically or by genetic testing to identify a predisposition to degenerative eye diseases. Examples of opthalmalogical disease includes Leber's Hereditary Optic Neuropathy. When symptoms such as elevated intraocular pressure, prior to the presentation of degenerative symptoms are treated with protective compounds, the degenerative disease can be forestalled and the symptoms minimized.

In an embodiment of the invention, patients who are receiving or are in need of anti-retroviral therapy for human immunodeficiency virus (HIV) infection may be identified, these patients are screened for susceptibility to a degenerative eye disease such as Leber's hereditary optic neuropathy, and such positive-testing patients may then be treated prophylatically or therapeutically with protective compounds.

In addition, subjects taking one or more medications known to perturb mitochondria, for example ethambutol, and/or medications known to cause optic neuropathy may be treated with a protective compound.

Protective compounds may be administered singly or in combinations of two or more protective compounds, with or without other active drugs, including without limitation, anti-glaucoma agents (for example, prostaglandins or prostanoids, carbonic anhydrase inhibitors, beta-adrenergic agonists and antagonists, alpha-adrenergic agonists, N-acetyl cysteine, glutathione, or other anti-glaucoma agents) known to those skilled in the art. Protective compounds also include enantiomers, diastereomers or racemic mixtures of the protective compounds as well as pharmaceutically acceptable salts of these compounds. Protective compounds may be delivered within any appropriate pharmaceutical formulation by oral delivery means, intravenously, subcutaneously, intramuscularly, intraocularly, transdermally, bucally, nasally, intracerebrally, intraspinally, topically (eg., eye drops), or any of a variety of novel alternative drug delivery systems including those currently marketed, or any other means that is appropriate to the compound(s) in question. Topical ophthalmic compositions are employed when the compounds are to be dosed topically. The preparation of topical ophthalmic compositions is well known in the art. Generally, topical ophthalmic compositions useful in the present invention are in the form of a solution, suspension, gel or formulated as part of a device, such as a collagen shield or other bioerodible or non-bioerodible device. Various excipients may be contained in the topical ophthalmic solutions, suspensions or gels of the present invention. For example, buffers (e.g., borate, carbonate, phosphate), tonicity agents (e.g., sodium chloride, potassium chloride, polyols), preservatives (e.g., polyquaterniums, polybiguanides, BAS), chelating agents (e.g., EDTA), viscosity enhancing agents (e.g., polyethoxylated glycols) and solubility agents (e.g., polyethoxylated castor oils, including polyoxl-35 castor oil, Polysorbate 20, 60 and 80; Pluronic.RTM. F-68, F-84 and P-103, or cyclodextrin) may be included in the topical ophthalmic compositions. A variety of gels may be useful in topical ophthalmic gel compositions of the present invention, including, but not limited to, carbomers, polyvinyl alcohol-borate complexes, or xanthan, gellan, or guar gums.

Topical ophthalmic bioerodible and non-bioerodible devices (e.g., conjunctival implant) (Weiner, A. L., Polymeric Drug Delivery Systems For the Eye, in Polymeric Site-specific Pharmacotherapy, Ed., A. J. Domb, John Wiley & Sons, pages 316-327 (1994) may be used for topical administration of protective compounds. Topical administration is suitable for facilitating the delivery of the protective compounds described herein to enable chronic treatment of the eye.

Protective compounds may also be delivered on a solid or semisolid scaffold, wherein delivery is accomplished by placing the support in a region of the eye selected from the group consisting of the eyelid, conjunctiva, sclera, retina, optic nerve sheath, an intraocular location and an intraorbital location. Additionally, protective compounds may be delivered slowly, over time, to the afflicted tissue of the eye through the use of contact lenses. This regimen is generally performed by first soaking the lenses in a protective compound, and then applying the contact lenses to the eye for normal wear.

Alternatively, protective compounds may be used with cultured cells or tissue maintained ex vivo for purposes of transplantation into one or more sites in the eye. In this instance, protective compound would enhance survival and viability of the tissue and increase the chances of a successful graft. Use of protective compounds in this context can be achieved with any of the available culturing or grafting procedures.

When the protective compounds are administered during intraocular, intracerebral or intraspinal surgical procedures, such as through retrobulbar or perocular injection, intraocular perfusion or injection, or intraspinal or intracerbral injection or perfusion, the use of irrigating solutions as vehicles are most preferred. The most basic irrigating solutions generally comprise sterile saline, or phosphate-buffered saline. More advanced irrigating solutions, however, are preferred. As used herein, the term “physiologically balanced irrigating solution” refers to a solution which is adapted to maintain the physical structure and function of tissues during invasive or noninvasive medical procedures. This type of solution typically contains electrolytes, such as sodium potassium, calcium, magnesium and/or chloride; an energy source, such as dextrose; and a-bicarbonate-buffer to maintain the pH of the solution at or near physiological levels. Various solutions of this type are known (e.g., Lactated Ringers Solution, BSS, RTM, BSS Plus RTM, Sterile Irrigating Solution, and Sterile Intraocular Irrigating Solution). Retrobulbar and periocular injections are useful techniques also known to those skilled in the art and are described in numerous publications including, for example, Ophthalmic Surgery: Principles of Practice, Ed., G. L. Spaeth, W. B. Sanders Co., Philadelphia, Pa., USA, pages 85-87 (1990).

Pharmaceutical compositions of the protective compounds can be formulated for systemic use using techniques well known in the art. Oral compositions are generally in the form of tablets, hard or soft gelatin capsules, suspension, granules, powders or other typical compositions and contain excipients typically present in such compositions. Methods for the preparation of such oral vehicles are well known by those skilled in the art. Parenteral administration compositions are generally be in the form of injectable solutions or suspensions. Methods for the preparation of such parenteral compositions are well known by those skilled in the art.

Example 1 provides results using an in vitro model of macular degeneration. Example 2 shows results using trabecular cells which are non-neuronal. The trabecular meshwork is involved in the regulation of aqueous humor homeostasis, critical for maintaining normal intraocular pressure. Abnormally high IOP is a significant risk factor for glaucoma. Example 3 provides in vivo results in a retinal ganglial cell degeneration model, a model for various ophthalmic diseases caused by, or resulting from degeneration of retinal ganglial cells. In Examples 1-5, polycyclic phenolic compounds were highly protective, demonstrating therapeutic utility.

All references cited herein are incorporated by reference.

### EXAMPLES

1. Administration of PAM and PACA leads to survival of retinal pigment epithelial cells subjected to conditions of hyperoxia in vitro.

The compounds used in this study, [2S-(2a, 4aα, 10aβ)]- 1,2,3,4,4a,9,10,10a-octahydro-7-hydroxy-2-methyl-2-phenanthrenemethanol (A, below) and [2S-(2a, 4aα, 10aβ)]- 1, 2, 3, 4, 4a, 9, 10, 10a-octahydro-7-hydroxy-2-methyl-2-phenanthrenecarboxaldehyde (B, below), were synthesized according to the procedures described in U.S. Pat. No. 4,874,891.

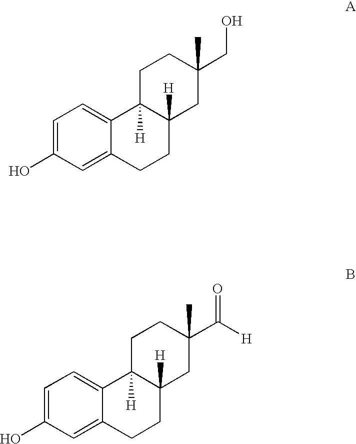

Human retinal pigment epithelial cells (RPE340 cells) were grown and subjected to conditions of hyperoxia according to Honda et al., (2001) Invest Ophthalmol Vis. Sci., Vol. 42, pp. 2139-44. The cells were subjected to either 40% O₂ or 20% O₂ (control) for the duration of the experiment. These conditions are known to induce apoptotic cell death.

Compounds A and B, above, were initially dissolved at 1 mg/ml in absolute ethanol and diluted in culture medium to a final concentration of 2 nM. To control for possible ethanol effects in the treated wells, all batches of media used here were supplemented with absolute ethanol at appropriate concentrations. Cells were incubated under hyperoxic conditions with either compound A, compound B or no compound added for a period of 5 days. Effects on cell viability and growth were monitored by Trypan blue exclusion. Our results show that compound A as well as compound B protect these cells from cell death in this model.

2. Administration of polycyclic phenolic compounds leads to survival of primary trabecular meshwork cells subjected to glutamate toxicity in vitro.

The compounds used-in this study, 2-(1-Adamantyl)-3-hydroxyestra-1, 3, 5 (10)-trien-17-one (C, below) and 2-(1-tert butyl)-3-hydroxyestra-1, 3, 5 (IO)-trien-17-one (D, below), were synthesized according to the procedures described in the literature.

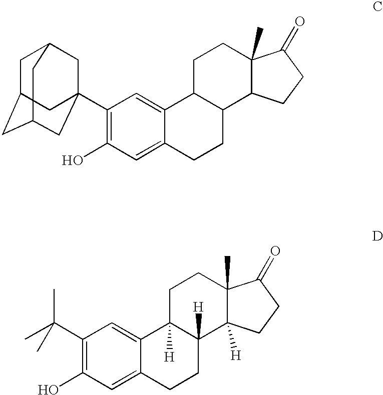

Trabecular meshwork cells used here were grown from fresh explants obtained from normal donors according to the procedures described in Agarwal et al., (1999) Exp Eye Res., Vol. 68, pp. 583 90 and Hogg et al., (2000) Invest Ophthalmol Vis. Sci., Vol. 41, pp. 1091-8. Cells were toxic conditions by exposure to hydrogen peroxide at 100 uM for 24 hours, which typically kills at least 50% of cells. With different batches of cells, the hydrogen peroxide concentration necessary to obtain 50% cell death frequently needs to be determined empirically. Trabecular cell viability was assessed using commercially available live-dead dyes. Other stains such as stains for DNA fragmentation or annexin binding are also suitable.

Compounds C and D, above, were initially dissolved at lmg/ml in absolute ethanol and diluted in culture medium to a final concentration of 2nM. To control for possible ethanol effects in the treated wells, all batches of media used here as were supplemented with absolute ethanol at appropriate concentrations. Cultured trabecular cells were incubated under toxic conditions with either compound C, compound D or no compound added for a period of 24 hours. Trabecular cell viability was assessed using commercially available live-dead dyes. Compounds C and D were both able to significantly improve survival under these conditions.

3. Intraocular administration of 17β-estradiol and ent-17β-estradiol leads to survival of axotomized retinal ganglion cells in adult rats.

The compounds used in this study were 17β-estradiol (E) and ent-17β-estradiol (F). 17β-estradiol was purchased from Steraloids, Inc. (Newport, R.I.) and ent-17β-estradiol (Chemistry Abstracts Registry Number, 3736-22-9) was synthesized according to the procedures described in WO 01/10430 A2, herein incorporated by reference.

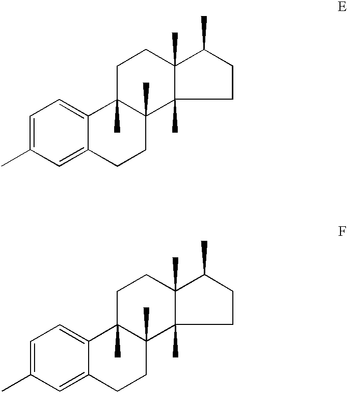

The drugs were dissolved in sesame oil (Penta) at 100 ug/ml. Adult female rats to be used in these experiments were first ovarectomized. Rats were then subjected to intraorbital transection of the optic nerve, a procedure that leads to progressive degeneration of the majority of retinal ganglion cells. Cells survive for a few days and then die abruptly such that by one week only 50% survive and by two weeks only 10% survive. Axotomy is well known to lead to the death of retinal ganglion cells by an apoptotic process, causing classical cytochemical alterations characteristic of programmed cell death.

From two days prior to eight days following optic nerve transactions, 17β-estradiol, ent-17β-estradiol, or appropriate amount of vehicle control sample was administered to the animals by a twice daily subcutaneous injection of compound in oil at 100 ug/ml (see above), such that the final concentration delivered was 100 ug/. If the protective compounds to be used are in a water soluble formulation, they can, alternatively, be delivered by a single intravitreal injection. Numerous alternative formulations, as described previously, are possible for administration of protective compounds.

After an additional 4 weeks, the retinal ganglial cells were labeled by retrograde staining with the carbocyanine dye, 4Di-10Asp, which was applied to the nerve stump. Three days later animals were sacrificed and surviving retinal cells were counted in retinal whole mounts. Animals treated with either 17β-estradiol or ent-17β-estradiol had a 3-fold greater number of surviving retinal ganglial cells compared to the controls.

Animal procedures and analytical techniques are described in detail in Mansour-Robaey et al., (1994) PNAS, Vol. 91, pp. 1632-6; Berkelaar et al., (1994) Neurosci, Vol. 14, pp. 4368-74; Peinado-Ramon et al., (1996) Invest Ophthalmol Vis. Sci., Vol. 37, pp. 489-500; Mey et al., (1993) Brain Res. Vol. 602, pp. 304-17.

4. PPCs Moderate Ca⁺² Induced ΔΨₘ Collapse in SHSY-5Y Neuroblastoma Cells and Transformed RGCs

Protective compounds useful for practicing exemplary embodiments of the invention may be derived from a wide variety of polycyclic phenolic compounds (PPCs). Previous published studies have shown that the naturally-occurring 17β-estradiol, its non-hormonal isomer, 17α-estradiol, and certain other non-hormonal synthetic analogs such as the complete enantiomer of 17β-E2 (ent17β-E2), are highly neuroprotective against glutamate toxicity in the rat hippocampal HT-22 cell line and in other in vitro models (Green, et al., 1997a,b; 2001; Simpkins, et al., 1997; Garcia-Segura, et al., 2001). These compounds also significantly reduce infarct volume in animal models of stroke in either pretreatment or post-treatment paradigms (Simpkins et al., 1997; Yang, et al., 2000a,b).

Surprisingly, we have observed that the cytoprotective effects of these compounds appear to be completely independent of hormonal activities; in fact, many of the non-hormonal estrogen analogs are more potent cytoprotectants than either the parental 17β-estradiol or its 100-fold less hormonal isomer, 17α-estradiol. Based on receptor binding-displacement assays, none of the analogs interacts with either of the classical estrogen receptors, ERα or ERβ, indicating the absence of hormonal activities and the associated health risks, an important consideration for a chronic glaucoma treatment for both males and females.

The neuroprotective activity of these compounds is dependent on the presence of a phenolic ring in the A position, and activity is lost if an acetate is substituted for the hydroxyl (Green, et al., 1997; Behl, et al., 1997). Structure-activity and mechanistic data suggest that these PPCs intercalate into cellular membranes, including the mitochondrial membranes, where they interrupt lipid peroxidation chain reactions that undermine membrane integrity (Dicko et al., 1999; Laing, et al., 2001; Wang, et al., 2001). Because mitochondrial function, and the necrosis or apoptosis that results from mitochondrial collapse, are so profoundly dependent upon the integrity of the inner mitochondrial membrane, the membrane-active PPCs are likely to promote mitochondrial integrity preferentially (Wang, et al., 2001). Significantly, the concentrations of these compounds required for neuroprotective effects in vivo are orders of magnitude lower than the concentrations required for ex vivo antioxidant effects, indicating a catalytic mechanism of action rather than a simple mass action antioxidant activity. For example, substantial cytoprotection with 17β-estradiol and 17α-estradiol in cell culture models is routinely achieved at doses as low as 0.2 nM (Green, et al., 1997), far below the concentrations required for classical radical scavenging activity. In accord with a catalytic model of function, the presence of glutathione in the culture medium lowers the EC50 of several of these PPCs for neuroprotection by at least 400-fold, suggesting a recycling mechanism involving glutathione (Green, et al., 1997).

In accord with a direct mitochondrial mechanism of action, 17β-estradiol, has been shown to preserve mitochondrial function in the presence of 3-nitropropionic acid, an inhibitor of oxidative phosphorylation that kills neurons in culture and in vivo (Wang, et al., 2001).

As a result of the teachings of exemplary embodiments of the present invention PPC libraries may be evaluated to assemble a collection of compounds optimized to retain neuroprotective activity, while eliminating potential hormonal activity, important considerations for the long-term treatment of glaucoma in both genders, especially with systemic formulations.

Mitochondrial activity of 17β-estradiol and 17α-estradiol.

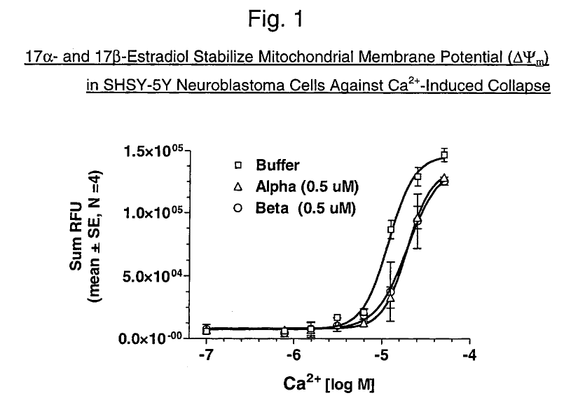

Without wishing to be bound to any particular theory, it is believed that the data indicate that the PPCs may exert their potent neuroprotective effects by preserving mitochondrial function, at least in part by stabilizing membrane structure during excessive Ca⁺² loading. As shown in FIG. 1, both 17β-estradiol and 17α-estradiol substantially increase the amount of Ca⁺² required to induce ΔΨₘ collapse in neuroblastoma cells. This is reflected by the EC₅₀ data associated with FIG. 1. These experiments suggest that at a given Ca⁺² load, a larger portion of the mitochondrial population in cells treated with these compounds retain ΔΨₘ, thereby maintaining cellular energetic and oxidative homeostasis, and hence forestalling necrosis or apoptosis.

4. 17β-Estradiol, 17α-Estradiol, and PPCs Moderate Ca⁺² Induced ΔΨₘ Collapse in SHSY-5Y Neuroblastoma Cells and Transformed RGCs

Without wishing to be bound to any particular theory, it is believed that the data indicate that the PPCs may exert their potent neuroprotective effects by preserving mitochondrial function, at least in part by stabilizing membrane structure during excessive Ca⁺² loading.

Mitochondrial effects in SHSY-5Y cells

As shown in FIG. 1, both 17β-estradiol and 17α-estradiol substantially increase the amount of Ca⁺² required to induce ΔΨₘ collapse in SHSY-5Y neuroblastoma cells. This is reflected by the right shift in the dose response curves compared to the control. These experiments suggest that at a given Ca⁺² load, a larger portion of the mitochondrial population in cells treated with these compounds retain ΔΨₘ, thereby maintaining cellular energetic and oxidative homeostasis, and hence forestalling necrosis or apoptosis.

With reference to FIG. 1, mitochondria rapidly take up exogenous Ca⁺² to a maximum value before undergoing an irreversible loss of ΔΨₘ. ΔΨₘ was monitored after exposure to Ca⁺² concentrations ranging from 0 to 50 uM using a fluorescence resonance energy transfer (FRET) assay (Dykens and Stout, 2001). Cells were preincubated with 17β-estradiol and 17α-estradiol at 0.5 uM for 2.5 hrs. prior to Ca⁺² challenge, which was imposed by adding Ca⁺² directly to cells that had been permeabilized with digitonin (0.008%) final concentration, 5 min). The response, calculated as area under the curve, was plotted versus log Ca⁺² concentration using sigmoid regression analysis. The EC50 values for both 17α-estradiol (18.68+0.3SE, N=4) and 17β-estradiol (19.44+0.5 SE, N =4) are significantly (P<0.01, covariance analysis) increased over controls (11.83+0.2 SE, N=4), with r2 correlation coefficients of 0.97, 0.94, and 0.99, respectively.

Mitochondrial effects in Transformed retinal ganglion cells

Transformed rat retinal ganglion cells (RGC-5 cells) were produced by transforming Sprague-Dawley rat retinal cells from postnatal day I rats with replication incompetent Ψ2 EIA virus and was originally established to help illuminate the basic mechanisms underlying RGC apoptosis in glaucoma (Krishnomoorthy, et al., 2001). The cells express the specific cellular marker, Thy-1, consistent with their retinal ganglion cell origin. Confirmation of Thy-1 expression was made by RT-PCR, immunocytochemistry and immunoblot analysis. The cells also express various neurotrophins and their receptors, and were shown to be dependent on the presence of trophic factors in the medium for survival. The toxicity response with glutamate has an EC50 of about 50 uM. Importantly, transformed RGC cells undergo TUNEL-positive apoptotic-mediated cell death upon serum deprivation, indicating the presence of classical apoptosis pathways induced by mitochondrial dysfunction and/or failure.

Possible mitochondrial responses of our compounds are evaluated in intact RGC-5 cells using an assay for mitochondrial ΔΨₘ which is based on fluorescence energy transfer (FRET) between two dyes that colocalize to the mitochondria. The FRET assay circumvents the confounding variables of plasma membrane potential and the low flourcscence efficiency typical of single potentiometric dyes (Dykens and Stout, 2001). Transformed retinal ganglion cells (RGC-5) were plated at a concentration of 60K cells/well twenty-four hours prior to the experiment. The cells were permeabilized in place with 0.08% digitonin. Cells were exposed to 17β-estradiol, 17α-estradiol, and a non-hormonal PPC, Compound H (see Example 5), for 5 minutes prior to the addition of Ca⁺².

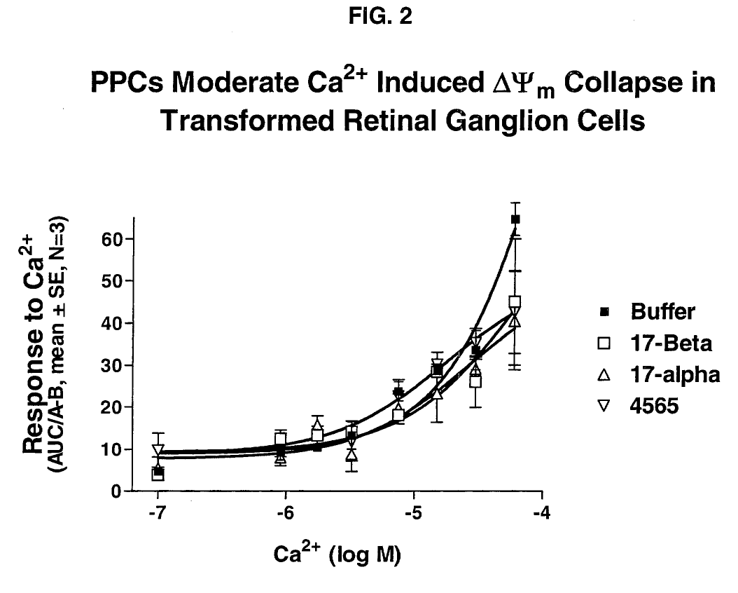

As shown in FIG. 2, when RGCs are exposed to Ca⁺² stress, mitochondrial membrane potential is lost, indicating permeability transition, as Ca⁺² concentration increases. However, compared to buffer-treated controls, in the presence of either 17β-estradiol and 17α-estradiol, or the non-hormonal PPC (Compound H, Example 5), the extent of mitochondrial collapse is moderated, as reflected by the lower curves for the three treatment groups. At the highest Ca⁺² concentration, the extent of mitochondrial failure is significantly lowered by 17b-E2, 17a-E2, and by Compound H (P<0.05 via ANOVA).

The excitation dye in the FRET ΔΨₘ assay is nonyl acridine orange (NAO). NAO is an exceptionally selective stain for cardiolipin, a lipid found intracellularly almost exclusively (>99%) in the mitochondrial inner membrane. Its staining of the inner membrane is independent of ΔΨₘ. The second dye is tetramethylrhodamine (TMR), a potentiometric dye that is sequestered into the mitochondrial matrix as a Nernstian function of ΔΨₘ and imposed concentration. It is the specificity of NAO staining for cardiolipin, combined with the absolute prerequisite for close proximity of both dyes, that allows this FRET assay to report ΔΨₘ without interference from plasma membrane potential.

Because uptake and retention of TMR depends on ΔΨₘ, at a given dye concentration, the extent of quenching of NAO by TMR reflects the magnitude of ΔΨₘ. Dissipation of ΔΨₘ, in response to uncouplers, elevated Ca₊₂ load, or respiratory uncouplers, results in efflux of TMR from the mitochondrion. The corresponding loss of proximity abolishes quenching of NAO, so that loss of ΔΨₘ is detected as an increase in NAO fluorescence.

5. Potent protective effect of non-hormonal polycyclic phenolic compounds

Many non-hormonal PPCs are more effective as neuroprotectants than 17β-estradiol. For instance, in this study, mouse HT-22 hippocampal cells were cultured in DMEM supplemented with 10% fetal calf serum. Cells were plated and simultaneously treated with glutamate (10 mM) and one of the test compounds individually, for instance compound H (also referred to as Compound 4565 in FIG. 2 and one of many protective PPCs disclosed in PCT publications WO02/36605 and WO00/63228), below or positive control 17β-estradiol (E), at various doses between 10 nM and 10 gM. After 16 hours, cell viability was determined using the calcein AM fluorescence (Excitation/Emission 485/530 nM) in a Bio-Tex Microplate Fluorescence Reader. Calculated EC50 values from a representative study were 0.37 μM of compound H compared to 2.20 μM of 17β-estradiol.

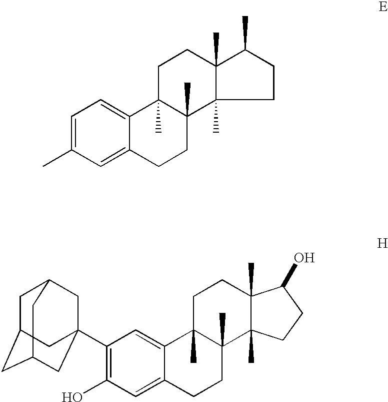

It should be understood that although Applicant has described exemplary embodiments of the present invention, it is expected and understood that one of ordinary skill in the art may make many alterations and modifications of the described embodiments and still remain within the scope of the invention as defined and interpreted solely by the claims which follow.

What is claimed is:

1. A method of reducing the frequency of cell death in ophthalmic tissue cells caused by physiological stressors, the method comprising the step of delivering at least one polycyclic phenolic compound to said ophthalmic tissue cells, said at least one polycyclic phenolic compound having at least one terminal phenol and at least one other cyclic group.

2. A method according to claim 1, wherein said stressors include necrosis and/or apoptosis.

3. A method according to claim 1, wherein said ophthalmic tissue cells comprise retinal ganglion cells.

4. A method according to claim 1, wherein the administration of the protective compound acts to normalizing intraocular pressure as well as stabilize mitochondrial structure and function.

5. A method according to claim 1, wherein the protective compounds is administered prophylactically to subjects identified as being at-risk but not yet showing signs of an ophthalmic disease, disorder or injury to prevent or delay onset of the disease.

6. A method according to claim 1, wherein the protective compound is co-administered to a subject together with one or more other medications known to perturb mitochondria and/or to cause optic neuropathy.

7. A method according to claim 1, wherein said ophthalmic tissue cells are retinal ganglion cells.

8. A method according to claim 1, wherein said cell death is caused by overexcitation of dicarboxylate receptors.

9. A method according to claim 1, wherein said one other cyclic group is terminal and is selected from the group consisting of substituted or unsubstituted aromatic or non-aromatic phenolics, phenanthrene, naphthalene, napthols, diphenyl, benzene, cyclohexane, 1,2-pyran, 1,4-Pyran, 1,2-pyrone, 1,4-pyrone, 1,2-dioxin, 1,3-dioxin (dihydro form), pyridine, pyridazine, pyrimidine, pyrazine, piperazine, s-triazine, as- triazine, v-triazine, 1,2,4-oxazine, 1,3,2-oxazine, 1,3,6-oxazine (pentoxazole), 1,2,6 oxazine, 1,4-oxazine, o-isoxazine, p-isoxazine, 1,2,5-oxathiazine, 1,2,6-oxathiazine, 1,4,2-oxadiazine, 1,3,5,2-oxadiazine, morpholine (tetrahydro-p-isoxazine).

10. A pharmaceutical composition for preventing or treating an ophthalmic disease, disorder or injury in a subject, comprising an effective dose of a polycyclic phenolic agent in a pharmaceutically acceptable formulation.

11. A pharmaceutical composition in accordance with claim 10 effective for reducing ophthalmic tissue cell death caused by exposure to excitatory neurotransmitters or xenobiotic dicarboxylate analogs.

12. A pharmaceutical composition in accordance with claim 10 effective for reducing ophthalmic tissue cell death caused by exposure to one of glutamate, quinolinate, kainite and ibotenate.

13. A pharmaceutical composition in accordance with claim 10 effective for reducing ophthalmic tissue cell death caused by decreased neurotrophic support.

14. A pharmaceutical composition in accordance with claim 10 effective for reducing ophthalmic tissue cell death caused by overexpression of nitric oxide synthase.

15. A pharmaceutical composition in accordance with claim 10 effective for reducing retinal ganglion cell death caused by increased hydrostatic pressure.

16. A pharmaceutical composition in accordance with claim 10 effective for reducing retinal ganglion cell death caused by decreased retrograde axoplasmic transport.

17. A pharmaceutical composition in accordance with claim 10 effective for reducing retinal ganglion cell death caused by free radical exposure.

18. A pharmaceutical composition in accordance with claim 10, effective for reducing retinal ganglion cell death caused by acute calcium ion loading.

19. A pharmaceutical composition in accordance with claim 10, wherein said composition is in a pharmaceutically acceptable vehicle
